(19) United States
(12) Patent Application Publication
(10) Pub. No.: US 2024/0238550 A1
(43) Pub. Date: Jul. 18, 2024

(54) METHOD AND SYSTEM FOR GENERATING A GAS MIXTURE

(71) Applicant: HydroSpire Inc., Framingham, MA (US)

(72) Inventors: Simon Gregory Stone, Arlington, MA (US); Linda Ann Tempelman, Lincoln, MA (US)

(21) Appl. No.: 18/416,803

(22) Filed: Jan. 18, 2024

Related U.S. Application Data
(60) Provisional application No. 63/439,788, filed on Jan. 18, 2023.

(51) Int. Cl. A61M 16/10; A61M 16/00; C25B 1/04; C25B 9/19; C25B 9/75; C25B 13/08

(52) U.S. Cl. CPC A61M 16/10; A61M 16/0003; C25B 1/04; C25B 9/19; C25B 9/75; C25B 13/08; A61M 2016/003

(57) ABSTRACT

Method and system for generating a gas mixture. The gas mixture may be, for example, a hydrogen-oxygen or hydrogen-air gas mixture, and may be used for topical or inspired therapeutic applications. In one embodiment, the system may include a water electrolyzer combined with an oxygen humidifier reservoir such that hydrogen produced at the cathode of the water electrolyzer mixes directly with oxygen gas that has been introduced into the oxygen humidifier reservoir, for example, by sparging. In another embodiment, the cathode manifold of the water electrolyzer may be plumbed in series with a pump and the oxygen humidifier reservoir such that hydrogen-entrained water may be mixed with oxygen that has been delivered, for example, via sparging, to the oxygen humidifier reservoir. In an exemplary application, the system may be incorporated into a ventilator circuit and may provide a breathing gas mixture containing hydrogen to treat ischemia/reperfusion injury of brain tissue.

### CROSS-REFERENCE TO RELATED APPLICATIONS

The present application claims the benefit under 35 U.S.C. 119(e) of U.S. Provisional Patent Application No. 63/439,788, inventors Simon Gregory Stone et al., filed Jan. 18, 2023, the disclosure of which is incorporated herein by reference.

### BACKGROUND OF THE INVENTION

The present invention relates generally to methods and systems for generating gas mixtures and relates more particularly to a novel method and system for generating a hydrogen-containing gas mixture, such as a gas mixture comprising oxygen and hydrogen gases. Such a gas mixture may be used, for example, to treat certain medical conditions including, but not limited to, ischemia-reperfusion injury, particularly ischemia-reperfusion injury to brain tissue.

Many diseases and traumas to the human body involve periods of ischemia, in which blood flow and, consequently, oxygen delivery to a part of the body are reduced. As can be appreciated, such periods of reduced blood flow and oxygen delivery can be deleterious to an affected part of the body. Unfortunately, the resumption of more normal blood flow following a period of ischemia, i.e., reperfusion, can also present a risk of injury to the affected part of the body. Such an injury, often referred to as ischemia/reperfusion injury (IRI), is attributable, at least in part, to the generation of reactive oxygen species that are produced during reperfusion, such reactive oxygen species being capable of causing a cascade of cellular processes that can result in chemical damage to DNA and, in some cases, can trigger apoptosis. A potential method of limiting the damage in the above situation is to administer one or more antioxidants that react quickly with the reactive oxygen species that are generated during reperfusion. One such antioxidant that has been identified for this purpose is molecular hydrogen, which may be administered to a patient by different modes of administration, such as by inhalation. Many publications have reported positively on the safety of hydrogen inhalation (and on other means of hydrogen administration), as well as the efficacy of hydrogen inhalation against various indications. See, for example, Ohsawa et al., “Hydrogen acts as a therapeutic antioxidant by selectively reducing cytotoxic oxygen radicals,” Nature Medicine, 13(6):688-694 (2007); and Cole et al., “Safety of Prolonged Inhalation of Hydrogen Gas in Air in Healthy Adults,” Critical Care Explorations, 3(10):e543 (2021), both of which are incorporated herein by reference.

A number of commercial products outside of the United States produce hydrogen-oxygen gas mixtures that are intended for inhalation. Some of these products are marketed as being suitable for treating various diseases whereas others of these products are marketed as being suitable for preventing certain diseases or disorders. Unfortunately, however, in many cases, these products generate a hydrogen-oxygen gas mixture at a ratio that results from direct water electrolysis, namely, 2H₂/1O₂. Such a 2:1 hydrogen-oxygen mixture is flammable and explosive and, therefore, presents an unacceptable risk in medical trauma, intensive care and emergency response settings. Consequently, products generating such gas mixtures are generally not suitable for use as medical devices in the United States, Europe, and many other countries. On the other hand, more dilute hydrogen-oxygen mixtures, such as 2-4 vol % H₂ in oxygen, are non-flammable and, therefore, intrinsically safe and have also shown clinical safety and preclinical efficacy in recently published studies. See, for example, Cole et al., “Perioperatively Inhaled Hydrogen Gas Diminishes Neurologic Injury Following Experimental Circulatory Arrest in Swine,” J Am Coll Cardiol Basic Trans Science, 4(2):176-87 (2019), which is incorporated by reference.

In view of the above, there is a clear need for a method and system that can be used to safely generate gas mixtures containing hydrogen gas, and there is also a clear need for a method and system that can be used to administer such hydrogen-containing gas mixtures to people, for example, via inhalation, for therapeutic and/or other purposes.

Additional documents that may be of interest may include the following, all of which are incorporated herein by reference: U.S. Pat. No. 11,414,765 B2, inventor Lin, issued Aug. 16, 2022; U.S. Pat. No. 11,105,003 B2, inventor Lin, issued Aug. 31, 2021; U.S. Pat. No. 10,265,665 B2, inventor Lin, issued Apr. 23, 2019; U.S. Pat. No. 9,050,278 B2, inventors Ohta et al., issued Jun. 9, 2015; U.S. Patent Application Publication No. US 2020/0368272 A1, inventors Satoh et al., published Nov. 26, 2020; U.S. Patent Application Publication No. US 2018/0295833 A1, inventors Hata et al., published Oct. 18, 2018; U.S. Patent Application Publication No. US 2015/0292091 A1, inventors Satoh et al., published Oct. 15, 2015; Japanese Patent Publication No. JP 6712924 B2, published Jun. 4, 2020; Japanese Patent Application Publication No. JP 2016000081 A, published Jan. 7, 2016; and Japanese Patent Application Publication No. JP 2023072784 A, published May 25, 2023.

### SUMMARY OF THE INVENTION

It is an object of the present invention to provide a new method and system for generating a gas mixture.

It is another object of the present invention to provide a method and system as described above that overcome at least some of the shortcomings of existing methods and systems for generating a gas mixture.

Therefore, according to one aspect of the invention, there is provided a system for generating a gas mixture, the system comprising (a) a water reservoir, the water reservoir comprising a volume of water, a first gas inlet through which a first delivered gas is delivered to the volume of water, and a gas outlet through which a gas mixture exits the water reservoir; and (b) a water electrolyzer, the water electrolyzer coupled to the water reservoir to receive water from the volume of water and to output a first generated gas that is added to the volume of water; (c) whereby the first delivered gas and the first generated gas mix in the volume of water to form the gas mixture and exit the water reservoir through the gas outlet.

In a more detailed feature of the invention, the first delivered gas may be oxygen, and the first generated gas may be hydrogen.

In a more detailed feature of the invention, the first delivered gas may be one of air and oxygen-enriched air, and the first generated gas may be hydrogen.

In a more detailed feature of the invention, at least a portion of the water electrolyzer may be positioned within the volume of water in the water reservoir.

In a more detailed feature of the invention, the first generated gas may be hydrogen, the water electrolyzer may comprise a cathode, and the cathode may be positioned within the volume of water in the water reservoir to output hydrogen directly into the volume of water.

In a more detailed feature of the invention, the water electrolyzer may further comprise an anode for outputting a second generated gas, and the system may further comprise a first fluid conduit for venting the second generated gas.

In a more detailed feature of the invention, the water electrolyzer may further comprise an anode for outputting a second generated gas, and the system may further comprise a second fluid conduit for conveying the second generated gas to the gas mixture at a location outside of the water reservoir.

In a more detailed feature of the invention, the water electrolyzer may be positioned entirely outside of the water reservoir.

In a more detailed feature of the invention, the system may further comprise a pump, and the pump may be fluidly coupled between the water reservoir and the water electrolyzer to pump water from the water reservoir to the water electrolyzer.

In a more detailed feature of the invention, the pump may be a cathode feed pump.

In a more detailed feature of the invention, the water electrolyzer may consist of a single electrolysis cell.

In a more detailed feature of the invention, the water electrolyzer may comprise a bipolar stack of electrolysis cells.

In a more detailed feature of the invention, the system may further comprise a bubble eliminator, and the bubble eliminator may be positioned in series between the water reservoir and the pump.

In a more detailed feature of the invention, the water electrolyzer may comprise a polymer electrolyte membrane, an anode operatively coupled at one face of the polymer electrolyte membrane, and a cathode operatively coupled to an opposing face of the polymer electrolyte membrane.

In a more detailed feature of the invention, the water reservoir may further comprise a second gas inlet through which a second delivered gas may be delivered to the volume of water.

In a more detailed feature of the invention, the second delivered gas may comprise at least one therapeutic gas selected from the group consisting of anesthesia and nitric oxide.

In a more detailed feature of the invention, the gas mixture may comprise hydrogen and oxygen, and the hydrogen concentration may not exceed 4 vol %.

In a more detailed feature of the invention, the system may further comprise a power supply for powering the water electrolyzer and a controller for controlling the output of the power supply.

In a more detailed feature of the invention, the system may further comprise a gas flow meter for measuring the flow of gas of the first delivered gas to the volume of water, and the gas flow meter may be operatively coupled to the controller.

In a more detailed feature of the invention, the system may further comprise a hydrogen gas sensor for measuring the hydrogen concentration of the gas mixture, and the hydrogen gas sensor may be operatively coupled to the controller.

According to another aspect of the invention, there is provided a ventilator system, the ventilator system comprising (a) an oxygen-containing gas supply, the oxygen-containing gas supply providing a quantity of an oxygen-containing gas; (b) a ventilator, the ventilator operatively coupled to the oxygen-containing gas supply to receive the oxygen-containing gas and to output the oxygen-containing gas; (c) the system for generating a gas mixture as described above, wherein the oxygen-containing gas is the first delivered gas and wherein the first generated gas is hydrogen gas, whereby the gas mixture comprises oxygen and hydrogen; and (d) a patient respiration interface device, the patient respiration interface device being operatively coupled to the ventilator and to the system for generating a gas mixture so that the gas mixture is administered to a patient via inhalation.

According to still another aspect of the invention, there is provided a method for generating a gas mixture, the method comprising the steps of (a) providing a water reservoir, the water reservoir comprising a volume of water, a first gas inlet through which a first delivered gas is delivered to the volume of water, and a gas outlet through which a gas mixture exits the water reservoir; (b) providing a water electrolyzer, the water electrolyzer coupled to the water reservoir to receive water from the volume of water and to output a first generated gas that is added to the volume of water; (c) delivering the first delivered gas to the volume of water; (d) operating the water electrolyzer to generate the first generated gas; (e) whereby the first delivered gas and the first generated gas mix in the volume of water to form the gas mixture and exit the water reservoir through the gas outlet.

For purposes of the present specification and claims, various relational terms like “top,” “bottom,” “proximal,” “distal,” “upper,” “lower,” “front,” and “rear” may be used to describe the present invention when said invention is positioned in or viewed from a given orientation. It is to be understood that, by altering the orientation of the invention, certain relational terms may need to be adjusted accordingly.

Additional objects, as well as aspects, features, and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawn to scale, and certain components may have undersized and/or oversized dimensions for purposes of explication. In the drawings wherein like reference numeral represents like parts:

FIG. 1 is a simplified schematic diagram of a first embodiment of a system for generating a gas mixture, the system being constructed according to the present invention;

FIG. 2 is a simplified section view of the water electrolyzer shown in FIG. 1;

FIG. 3 is a partly exploded perspective view of one embodiment of the combined water reservoir and water electrolyzer shown in FIG. 1;

FIG. 4 is a simplified schematic diagram of a second embodiment of a system for generating a gas mixture, the system being constructed according to the present invention;

FIG. 5 is a simplified schematic diagram of a third embodiment of a system for generating a gas mixture, the system being constructed according to the present invention;

FIG. 6 is a simplified schematic diagram of a fourth embodiment of a system for generating a gas mixture, the system being constructed according to the present invention;

FIG. 7 is a simplified schematic diagram of a fifth embodiment of a system for generating a gas mixture, the system being constructed according to the present invention;

FIG. 8 is a simplified schematic diagram of a sixth embodiment of a system for generating a gas mixture, the system being constructed according to the present invention;

FIG. 9 is a schematic diagram of a first embodiment of a baffled fluid conduit constructed according to the present invention;

FIG. 10 is a simplified schematic diagram of a first embodiment of a ventilator circuit constructed according to the present invention;

FIG. 11 is a simplified schematic diagram of a second embodiment of a ventilator circuit constructed according to the present invention;

FIG. 12 is a timeplot of the hydrogen concentration, oxygen flow rate, and cell current during constant-current electrolysis of water using the setup described in Example 1;

FIG. 13 is a graph showing the measured hydrogen concentration at the reservoir outlet during constant electrolysis at 2.7 A and 1.0 SLPM sparged oxygen delivery to electrolyzer reservoir in the setup of Example 1; and

FIG. 14 is a polarization plot for the setup in operation as described in Example 1.

### DETAILED DESCRIPTION OF THE INVENTION

The present invention is directed at a novel method and system for generating a gas mixture. Such a gas mixture may be, but is not limited to, a hydrogen-containing gas mixture, such as a gas mixture comprising hydrogen and oxygen gases or a gas mixture comprising hydrogen gas and air. The aforementioned gas mixture may be suitable for administration, for example, by inhalation and/or one or more other modes of administration (e.g., topical administration), to a person for therapeutic and/or preventive medical or health purposes. For example, such a gas mixture may be administered by inhalation to a person suffering from an ischemia/reperfusion injury of brain tissue or other tissue in order to mitigate the effects of such an injury. Such administration by inhalation may be achieved, for example, by incorporating the gas mixture generating system into a respiratory or breathing apparatus, wherein the respiratory or breathing apparatus may additionally include, for example, a ventilator and a mask or breathing tube.

More specifically, according to at least one embodiment, oxygen gas from a medical supply source (i.e., feed oxygen or “delivered gas”) may be mixed with hydrogen that is generated via electrolysis, preferably via the electrolysis of water (i.e., “generated gas”). The feed oxygen may be an oxygen-rich stream of about 21-100 volume percent oxygen, with the balance composed of nitrogen; however, the feed oxygen may also contain other therapeutic or inert gases or vapors. In at least one embodiment, electrolysis may be effected using a proton exchange membrane (PEM) water electrolyzer, wherein said PEM water electrolyzer may comprise two catalyzed electrode systems (electrocatalyst films) separated by an ionically conductive proton exchange membrane. Using a direct current, the aforementioned PEM water electrolyzer may electrolytically produce high purity oxygen and hydrogen gases as discrete product streams whose separation is substantially maintained by the ionically conductive proton exchange membrane. Electrically conductive components and seals may surround the electrode systems to allow the conduction of electricity through the cell with low ohmic resistance, and fluid (liquid and gas) inputs and outputs may be well directed across the electrocatalyst surface without leaks, across the proton exchange membrane, or to the ambient environment. Hydrogen may be generated at the electrolyzer cathode, which may be submerged in a suitable type of water (e.g., sterile, deionized water), and such hydrogen may exist therein in the dissolved state and/or in entrained bubbles. The small amount of electrolytic oxygen that may be produced by the electrolyzer may be vented for convenience or, optionally, may be included in the water reservoir exit stream. The mixing of the generated hydrogen with feed oxygen may be accomplished in both liquid and gaseous phases in a water reservoir that may be similar in many respects to a ventilator humidifier used for oxygen humidification, such as a ventilator humidifier unit of the type that is commercially available as Medline Hudson RCI bubble humidifier (Medline Industries, LP, Uxbridge, MA). According to the foregoing mixing technique, feed oxygen bubbles, which may be generated, for example, at a sparger, may coalesce with hydrogen bubbles produced by electrolysis to accomplish mixing, and dissolved gases may also equilibrate at each bubble-water interface. This rapid mixing method with its fine gas bubbles may decrease the incidence (in time and space) of gas mixtures exceeding 4 vol % H₂ (i.e., the lower flammable limit). In addition, having the mixing occur surrounded by water may minimize the probability of ignition in the mixing area. This method may safely produce outlet streams of any concentration less than 4 vol % of hydrogen in air or oxygen.

In at least one embodiment, the electrolyzer may be of a type other than a PEM electrolyzer. For example, the electrolyzer may be a liquid electrolyzer of the type in which an anode and a cathode of any of a variety of shapes known in the art may be submerged in a liquid water reservoir that additionally contains sufficient electrolytes to complete the circuit and that leads to water electrolysis with hydrogen at the cathode and oxygen at the anode bubbling into the reservoir. In such an embodiment, feed oxygen may additionally be added to the reservoir, for example, by sparging, in order to generate a desired mixture of oxygen and hydrogen gases.

In at least one embodiment, the system may also comprise a controller to control the rate of electrolysis to produce a safe hydrogen-oxygen mixture composition. Such a controller may set the electrolysis rate (applied direct current) based on input oxygen flow and desired hydrogen concentration. The current setpoint may be calculated from a modification of Faraday's Law and may define a pseudo-open loop control scheme:

$$i = n \times F \times {f}_{hyd} / {V}_{m} \qquad ( Eq . 1 )$$

wherein i is the current setpoint (amperes), n is the number of electrons transferred in the hydrogen evolution reaction (2 electrons per molecule of hydrogen gas), F is Faraday's constant (96,485 A-s/mol e⁻), find is the desired hydrogen flow rate (cm³/s) and Vₘ is the molar volume from the ideal gas law (24,100 cm³/mol gas). The desired hydrogen flow rate may be calculated from the oxygen flow rate and the desired hydrogen concentration as:

$${f}_{hyd} = {f}_{oxy} / [ ( 1 / {X}_{H 2} ) - 1 ] \qquad ( Eq . 2 )$$

wherein f_{oxy} is the input oxygen flow and X_{H2} is the desired hydrogen concentration.

In the foregoing embodiment, only one sensor may be needed (oxygen flow rate), but errors in flow controller function or incomplete oxygen delivery to a sparger may result in output hydrogen concentrations above the flammable limit. To mitigate against this risk, a close-loop approach may be utilized which operates to control the desired hydrogen concentration at the target level via a hydrogen sensor at the outlet. In such an embodiment, a proportional-integral-derivative control algorithm may be tuned to accurately and responsively manage output hydrogen concentration within narrow setpoint limits. Other suitable control algorithms may be applied. The system also may include a source of power, which may be wall AC current converted to DC current or a DC power supply. The voltage for electrolysis is preferably supplied at greater than or equal to 1.3V, and the current is preferably supplied at the level for the desired hydrogen production rate as described above.

In at least one embodiment, the system may include a water reservoir similar in many respects to a humidifier reservoir of the type that is used in commercial ventilators. Such a water reservoir may be warmed to about 35° C.-37° C. to achieve a desired humidity level for inhalation. For additional mitigation of flame, propagation layers of high surface area metal (sometimes called “flame arrestors”) may also be included in the reservoir or in the gas outlet exiting the reservoir to prevent flame front movement by temperature regulation.

In at least one embodiment, the system may comprise a water electrolyzer integrated with a water reservoir that is similar in many respects to a humidifier reservoir of a ventilator. In such an embodiment, hydrogen generated at the cathode of the water electrolyzer may be accomplished at the bottom face of the water reservoir where the water electrolyzer may be built in. The rate of hydrogen-oxygen mixing in the water reservoir may be determined by the convection properties of the two-phase fluid in the bubbly flow regime. The convection in this case may be dominated by the buoyancy of gases during simultaneous hydrogen evolution and oxygen sparging. The sparging of gas (oxygen, hydrogen, or other) may be accomplished by any method known in the art, such as fritted or porous ceramics, metals, glass or polymers. The hydrogen sparging may also be accomplished by the fine bubble formation that occurs naturally at the cathode of the electrolyzer directly into the water reservoir. The mixing of gases in the water reservoir may include one or more mechanisms or combinations of mechanisms including bubbles of gas phase gas joining with any combination of oxygen bubbles, hydrogen bubbles, or mixed gas bubbles combining with any of those bubble types. The water reservoir may alternatively or additionally be convectively stirred or mixed by any mechanism known in the art to aid in mixing. Another mechanism may comprise the dissolving of either or both gases into the soluble phase, mixing of those dissolved molecules, and those dissolved gases returning to the gas phase joining with gases in a gas phase bubble. Multiple gas bubbles may exit the liquid phase of the water reservoir and combine in the gaseous headspace of the water reservoir and exit the water reservoir via an exit conduit. The water reservoir geometry or materials may be designed to enhance mixing, gas solubility, or bubble coalescence in the liquid phase or bubble recombination in the headspace. The exit conduit may be designed to exclude liquid phase materials from leaving the water reservoir via the use of vapor phase permeable materials (such as GORE-TEX® expanded polytetrafluoroethylene (W. L. Gore & Associates, Inc. (Newark, DE)) or other porous materials. The exit conduit may include alternating baffling to trap liquids and to promote the return of liquids to the water reservoir or other methods to prevent rainout, such as those disclosed in U.S. Patent Application Publication No. US 2022/0168533 A1, inventors Malouf et al., issued Jun. 2, 2022, which is incorporated herein by reference.

Alternatively, in at least one embodiment, the same current control approaches as described above may be utilized, but the hydrogen-oxygen mixing may accomplished in a volume of recirculating water stream maintained by a pump from the water reservoir (where oxygen again may be introduced by sparging) through the electrolyzer cathode. While there still may be some bubbly flow characteristic in this system, convection may be further promoted by the pumping action, and the kinetics of mixing may be faster. This configuration may be particularly promising for larger therapeutic applications as the electrolyzer may be configured as a bipolar stack; consequently, its gas output may be magnified by cell number in the stack with a linear increase in applied voltage. Stacked bipolar cells are generally the best manner of increasing output due to the minimal increase in electrolyzer volume and cost, as well as the modest increase in power supply capability; power supplies and current distribution in the electrochemical system may become substantially more costly when output is scaled with monopolar area. Water flow in this configuration may be directed to the cathode manifold, as opposed to the anode-feed method typical of commercial water electrolyzers. Because of this, there may be a current limit to be avoided due to the need for water on the anode. Water must diffuse from the cathode across the membrane to the anode to support the electrolysis rate, and this diffusion may compete with electro-osmotic drag of water from the anode to the cathode, a pseudo-convective process. Nevertheless, with commercial membranes at or near room temperature, current densities of greater than 500 mA/cm² electrocatalyst area may be sustained stably in the steady state, i.e. with no voltage increase.

In at least one embodiment, the electrolytic current path may be incorporated directly into the water reservoir volume and therein may create separated or mixed hydrogen and oxygen gas streams. Catalyzed electrodes may be immersed in the aqueous electrolyte solution, and the medical oxygen feed may be sparged into the water volume below the electrodes to ensure hydrogen dilution. The electrolyte may consist of or comprise a salt (e.g., sodium sulfate), an acid (e.g., sulfuric acid), a base (e.g., potassium hydroxide), or a mixture thereof, and the aqueous solution of electrolytes may be of a composition and concentration sufficient to ensure that conductivity, gas purity, and oxygen humidity requirements are satisfied. One or both electrodes optionally may be integrated with the wall of the water reservoir.

In at least one embodiment, the system of the present invention may be used as a subsystem in a respiration or ventilation circuit for natural or assisted breathing for a human and/or animal. More broadly, the gas mixture generated using the present invention may be used for various human, veterinary, life science and/or industrial applications. For example, the system may be used in various life science settings where living cells may benefit from a particular hydrogen-containing gas mixture. More specifically, the system of the present invention may be used, for example, as a subsystem in a humidified cell culture incubator to add hydrogen to a standard or non-standard mixture of oxygen, carbon dioxide, and vapor phase water. The system of the present invention may also be used as a subsystem for generating a mixture of hydrogen and oxygen for a pressurized, bottled gas mixture where humidity may be retained or may be eliminated by various dryers known in the art. The system of the present invention may also be used as a subsystem for a veterinary cage system where a small space (e.g., an animal cage) may have a modified atmosphere containing a mixture of hydrogen and oxygen, with or without additional gases.

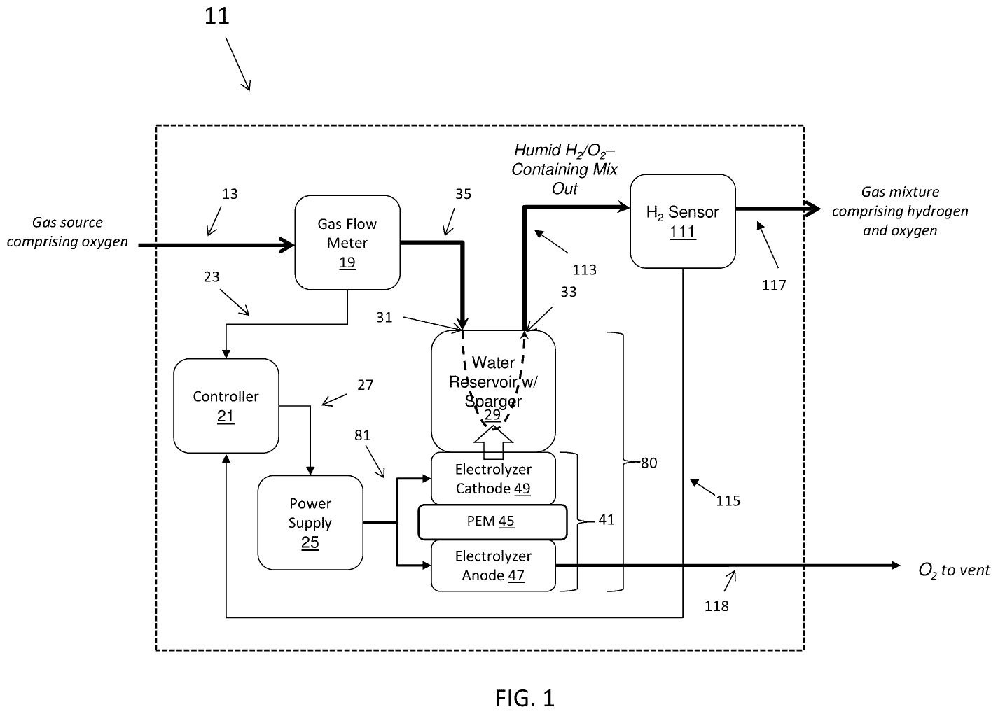

Referring now to FIG. 1, there is shown a simplified schematic representation of a first embodiment of a system for generating a gas mixture, the system being constructed according to the present invention and being represented generally by reference numeral 11. Details of system 11 that are discussed elsewhere in this application or that are not critical to an understanding of the invention may be omitted from FIG. 1 or the accompanying description herein or may be shown in FIG. 1 and/or described herein in a simplified manner.

System 11, which may be used, for example, to generate a gas mixture containing hydrogen and oxygen gases, may comprise a fluid conduit 13. Fluid conduit 13, which may consist of or comprise a tube or other structure well-suited for conveying one or more fluids axially therethrough, may have an inlet end and an outlet end. The inlet end may be fluidly coupled to an outlet of a gas source comprising oxygen. The gas source comprising oxygen may be any one or more of a variety of oxygen-containing gas sources and may consist of or comprise one or more conventional gas sources comprising oxygen gas, such as, but not limited to, pure oxygen, air, or oxygen-enriched air. For example, the gas source comprising oxygen may comprise a ventilator that is, in turn, coupled to an outlet of an oxygen gas supply, an air supply or a supply of oxygen-enriched air. Alternatively, the gas source comprising oxygen may comprise one or more gas tanks containing pure oxygen gas and/or one or more gas tanks containing a mixture of oxygen gas and one or more additional gases, wherein said one or more additional gases may include, for example, one or more inert gases, such as nitrogen gas, and/or one or more therapeutic gases, such as anesthesia. Alternatively, the gas source comprising oxygen may comprise a humidifier that is, itself, coupled to the outlet of the above-described ventilator or the above-described one or more gas tanks.

System 11 may further comprise a gas flow meter 19, wherein the outlet end of fluid conduit 13 may be fluidly coupled to an inlet of gas flow meter 19. Gas flow meter 19, which may be conventional, may be used to measure, either directly or indirectly, the amount of oxygen gas that is being inputted into system 11. In this manner, as will be discussed further below, an appropriate amount of hydrogen gas may be generated by system 11 and mixed with the aforementioned measured amount of oxygen gas.

System 11 may further comprise a controller 21, which may be conventional. The information that is collected by gas flow meter 19 may be transmitted to controller 21 via an electrical conduit 23, which may comprise an electrically-conductive wire or similarly suitable structure. Electrical conduit 23 may be electrically coupled at a first end to gas flow meter 19 and at a second end to controller 21. Controller 21, in turn, may be electrically coupled to a power supply 25, which may be, for example, a direct current power supply, via an electrical conduit 27, which may comprise an electrically-conductive wire or similarly suitable structure. Electrical conduit 27 may be coupled at a first end to controller 21 and at a second end to power supply 25. In this manner, controller 21 may be used to control the current that is emitted from power supply 25 and, as will become apparent below, the amount of hydrogen that is generated by system 11. (There is a generally linear relationship between current and the amount of hydrogen gas that is generated). Although not shown, controller 21 may include its own power supply or may derive power from power supply 25 through a suitable electrical conduit.

System 11 may further comprise a water reservoir 29. Water reservoir 29, which may be similar in many respects to a conventional humidifier of the type found in a ventilator circuit, may comprise a container adapted to receive a volume of water (the volume of water not being shown in FIG. 1). The container may comprise a first inlet 31 and a first outlet 33. The first inlet 31 may be fluidly coupled to an outlet of gas flow meter 19 via a fluid conduit 35, wherein fluid conduit 35 may consist of or comprise a tube or other structure well-suited for conveying one or more fluids axially therethrough. Accordingly, in this manner, the oxygen-containing gas passing through gas flow meter 19 may be inputted into water reservoir 29 through first inlet 31.

It is to be understood that, although first inlet 31 is shown in FIG. 1 as being positioned at the top of water reservoir 29, water reservoir 29 is typically not filled to capacity. Accordingly, first inlet 31 may be positioned on water reservoir 29 at a location that is below the water level of the volume of water that is disposed within water reservoir 29. Alternatively, first inlet 31 may be positioned at the top of water reservoir 29, but an inlet tube may be inserted through first inlet 31 to convey the oxygen-containing gas to a point that is below the water level of water reservoir 29. In this manner, the oxygen-containing gas that is dispensed into water reservoir 29 may be inputted directly into the volume of water, thereby being dispersed within the volume of water (and preferably creating gas bubbles in the volume of water), as opposed to being inputted directly into a headspace above the volume of water. Additionally, although not shown, a sparger may be positioned within water reservoir 29 at or near first inlet 31. Such a sparger, which may be conventional, may be used to produce small gas bubbles of the oxygen-containing gas within the volume of water. In the absence of such a sparger, larger gas bubbles may be produced, which may be less desirable for mixing.

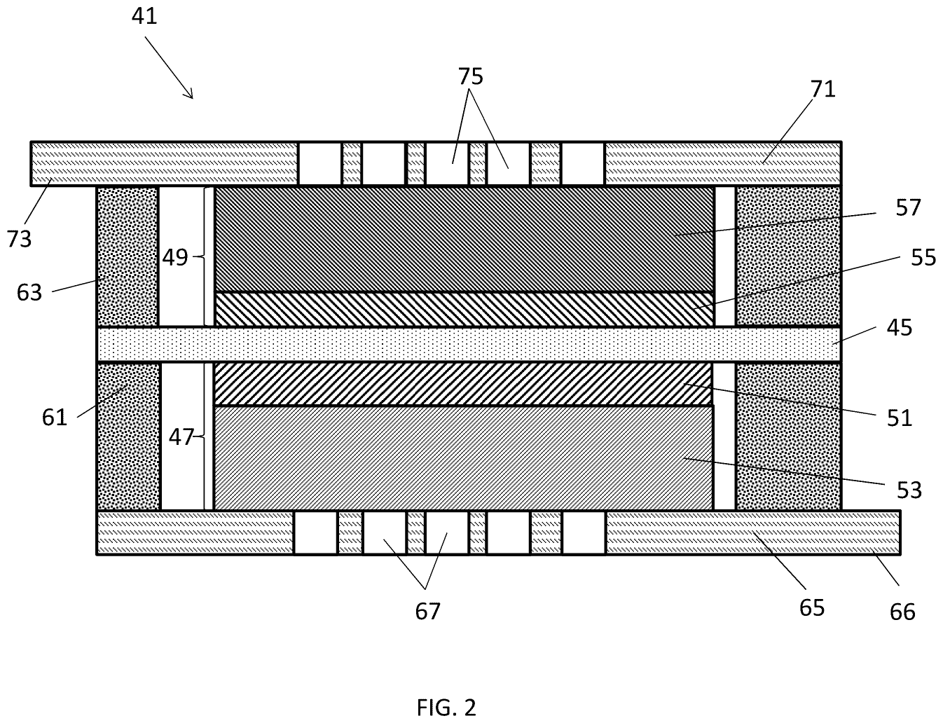

System 11 may further comprise a water electrolyzer 41, which is also shown in FIG. 2. In the present embodiment, water electrolyzer 41 may be a water electrolyzer of the type comprising a polymer electrolyte membrane (PEM), said polymer electrolyte membrane also sometimes referred to as a proton exchange membrane. Water electrolyzer 41 may be similar in many respects to conventional PEM water electrolyzers, such as, but not limited to, U.S. Pat. No. 9,357,764 B2, inventors Tempelman et al., Jun. 7, 2016, and U.S. Patent Application Publication No. US 2022/0054318 A1, inventors Schwenk et al., published Feb. 24, 2022, both of which are incorporated herein by reference.

Water electrolyzer 41 may comprise a polymer electrolyte membrane 45, an anode 47, and a cathode 49.

Polymer electrolyte membrane 45 is preferably a non-porous, ionically-conductive, electrically-non-conductive, liquid permeable and substantially gas-impermeable membrane. Polymer electrolyte membrane 45 may consist of or comprise a homogeneous perfluorosulfonic acid (PFSA) polymer. Said PFSA polymer may be formed by the copolymerization of tetrafluoroethylene and perfluorovinylether sulfonic acid. See e.g., U.S. Pat. No. 3,282,875, inventors Connolly et al., issued Nov. 1, 1966; U.S. Pat. No. 4,470,889, inventors Ezzell et. al., issued Sep. 11, 1984; U.S. Pat. No. 4,478,695, inventors Ezzell et. al., issued Oct. 23, 1984; and U.S. Pat. No. 6,492,431, inventor Cisar, issued Dec. 10, 2002, all of which are incorporated herein by reference in their entireties. A commercial embodiment of a PFSA polymer electrolyte membrane is manufactured by The Chemours Company FC, LLC (Fayetteville, N.C.) as NAFION™ extrusion cast PFSA polymer membrane.

Polymer electrolyte membrane 45 may be a generally planar unitary structure in the form of a continuous film or sheet. In the present embodiment, when viewed from above or below, polymer electrolyte membrane 45 may have a general circular shape. Moreover, the overall shape of water electrolyzer 41, when viewed from above or below, may correspond generally to the shape of polymer electrolyte membrane 45. However, it is to be understood that polymer electrolyte membrane 45, as well as water electrolyzer 41 as a whole, is not limited to a generally circular shape and may have a generally rectangular, annular, or other suitable shape.

Anode 47 and cathode 49 may be positioned along two opposing major faces of polymer electrolyte membrane 45. For reasons to become apparent from the description below, anode 47 is shown in the present embodiment to be positioned along the bottom face of polymer electrolyte membrane 45, and cathode 49 is shown positioned along the top face of polymer electrolyte membrane 45.

As seen best in FIG. 2, anode 47 may comprise an anode electrocatalyst layer 51 and an anode support 53. Anode electrocatalyst layer 51 may be positioned in direct contact with polymer electrolyte membrane 45, and, in the present embodiment, is shown as being positioned directly below and in contact with the bottom side of polymer electrolyte membrane 45. Anode electrocatalyst layer 51 defines the electrochemically active area of anode 47 and preferably is sufficiently porous and electrically- and ionically-conductive to sustain a high rate of surface oxidation reaction. Anode electrocatalyst layer 51, which may be an anode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within anode electrocatalyst layer 51 along with a binder, which is preferably ionically-conductive, to provide mechanical fixation.

Anode support 53, which may be an anode support of the type conventionally used in a PEM-based water electrolyzer and may be, for example, a film or sheet of porous titanium, preferably is sufficiently porous to allow fluid (gas and/or liquid) to pass freely therethrough. To this end, anode support 53 may have pore sizes on the order of, for example, approximately 0.001-0.5 mm. In addition, anode support 53 is preferably electrically-conductive to provide electrical connectivity between anode electrocatalyst layer 51 and an anode current collector to be discussed below. Anode support 53 is also preferably ionically-non-conductive. Anode support 53 may be positioned in direct contact with anode electrocatalyst layer 51 and, in the present embodiment, is shown as being positioned directly below anode electrocatalyst layer 51 such that anode electrocatalyst layer 51 may be sandwiched between and in contact with polymer electrolyte membrane 45 and anode support 53. Anode support 53 may be dimensioned to entirely cover a surface (e.g., the bottom surface) of anode electrocatalyst layer 51, and, in fact, anode 47 may be fabricated by depositing anode electrocatalyst layer 51 on anode support 53.

Cathode 49 may comprise a cathode electrocatalyst layer 55 and a cathode support 57. Cathode electrocatalyst layer 55 may be positioned in direct contact with polymer electrolyte membrane 45, and, in the present embodiment, is shown as being positioned directly above and in contact with the top of polymer electrolyte membrane 45. Cathode electrocatalyst layer 55 defines the electrochemically active area of cathode 49 and preferably is sufficiently porous and electrically- and ionically-conductive to sustain a high rate of surface reduction reaction. Cathode electrocatalyst layer 55, which may be a cathode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within cathode electrocatalyst layer 55 along with a binder, which is preferably ionically-conductive, to provide mechanical fixation. The reactants and products involved at anode 47 and cathode 49 may implicate ionic species that are mobile throughout the electroactive surface; therefore, an ionically-conductive medium comprising polymer electrolyte membrane 45 and, optionally, one or more ionically-conductive catalyst binders in electrocatalyst layers 51 and 55 may couple the electrodes and may allow ions to flow in support of the overall reaction electrochemistry.

Cathode support 57, which may be a cathode support of the type conventionally used in a PEM-based water electrolyzer and may be, for example, a film or sheet of porous carbon, preferably is sufficiently porous to allow fluid (gas and/or liquid) transfer freely therethrough. To this end, cathode support 57 may have pore sizes on the order of, or example, approximately 0.001-0.5 mm. In addition, cathode support 57 is electrically-conductive to provide electrical connectivity between cathode electrocatalyst layer 55 and a cathode current collector to be discussed below. Cathode support 57 is also preferably ionically-non-conductive. Cathode support 57 may be positioned in direct contact with cathode electrocatalyst layer 55 and, in the present embodiment, is shown as being positioned directly above cathode electrocatalyst layer 55 such that cathode electrocatalyst layer 55 may be sandwiched between and in contact with polymer electrolyte membrane 45 and cathode support 57. Cathode support 57 may be dimensioned to entirely cover a surface (e.g., the top surface) cathode electrocatalyst layer 55, and, in fact, cathode 49 may be fabricated by depositing cathode electrocatalyst layer 55 on cathode support 57.

The combination of polymer electrolyte membrane 45, anode 47, and cathode 49, or the combination of polymer electrolyte membrane 45, anode electrocatalyst layer 51, and cathode electrocatalyst layer 55 may be regarded collectively as a membrane-electrode assembly (MEA).

Water electrolyzer 41 may further comprise an anode seal 61 and a cathode seal 63. Anode seal 61, which may be an anode seal of the type conventionally used in a PEM-based water electrolyzer, may be a generally annular or frame-like member mounted around the periphery of anode 47 in a fluid-tight manner. (Anode seal 61 may be positioned in direct contact with the periphery of anode 47 or there may be a small gap between anode seal 61 and the periphery of anode 47 to facilitate assembly.) Anode seal 61, which may be made of polytetrafluoroethylene (PTFE), ethylene-propylene-diene-monomer (EPDM) rubber, or another similarly suitable material, may be ionically-non-conductive and electrically non-conductive. Anode seal 61 may also be non-porous and fluid-impermeable.

Cathode seal 63, which may be a cathode seal of the type conventionally used in a PEM-based water electrolyzer, may be a generally annular or frame-like member mounted around the periphery of cathode 49 in a fluid-tight manner. (Cathode seal 63 may be positioned in direct contact with the periphery of cathode 49 or there may be a small gap between cathode seal 63 and the periphery of cathode 49 to facilitate assembly.) Cathode seal 63, which may be made of polytetrafluoroethylene (PTFE), ethylene-propylene-diene-monomer (EPDM) rubber, or another similarly suitable material, may be ionically-non-conductive and electrically-non-conductive. Cathode seal 63 may also be non-porous and fluid-impermeable.

In the present embodiment, anode 47 and anode seal 61 may be dimensioned to jointly match the footprint of the bottom surface of polymer electrolyte membrane 45. In addition, cathode 49 and cathode seal 63 may also be dimensioned to jointly match the footprint of the top surface of polymer electrolyte membrane 45. Notwithstanding the above, it is to be understood that the footprints of the foregoing components may be varied from what is described above.

Water electrolyzer 41 may further comprise an anode current collector 65. Anode current collector 65 may be similar to an anode current collector of the type conventionally used in a PEM-based water electrolyzer and may comprise, for example, a platinum-coated titanium sheet. When viewed from below, anode current collector 65 may have a footprint that substantially matches the collective footprints of anode 47 and anode seal 61, except that anode current collector 65 may additionally comprise a tab 66 that may extend radially outwardly a short distance beyond the periphery of anode seal 61 and that may be used as a terminal. Anode current collector 65 may also comprise a plurality of through holes 67, through which oxygen generated at anode 47 may pass.

Water electrolyzer 41 may further comprise a cathode current collector 71, which may comprise a cathode current collector of the type conventionally used in a PEM-based water electrolyzer and may be, for example, a platinum-coated titanium sheet. When viewed from below, cathode current collector 71 may have a footprint that substantially matches the collective footprints of cathode 49 and cathode seal 63, except that cathode current collector 71 may additionally comprise a tab 73 that may extend radially outwardly a short distance beyond the periphery of cathode seal 63 and that may be used as a terminal. Cathode current collector 71 may also comprise a plurality of through holes 75, through which hydrogen generated at cathode 49 may pass.

Although not shown, water electrolyzer 41 may further comprise other components commonly found in conventional PEM-based water electrolyzers. For example, the static forces upon water electrolyzer 41 that may be required to compress anode seal 61 and cathode seal 63 to sustain good electrical contact of the serial components of water electrolyzer 41 and to achieve good sealing of the cell perimeter may be established and maintained using a variety of conventional fixturing or joining implements and techniques about the internal or external periphery of the assembly. Such implements may include, for instance, fasteners (e.g., screws, rivets, etc.) which may clamp endplates at either end of the serial components, or adhesives, cements, or welds which cohere the elements together in the seal region. Such implements and techniques are known to those of ordinary skill in the art.

Referring back now to FIG. 1, it can be seen that water electrolyzer 41 may be electrically coupled to power supply 25 via an electrical conduit 81, which may comprise one or more wires or similarly suitable structure. Electrical conduit 81 may be coupled to anode 47 (via current collector 65) and to cathode 49 (via current collector 71). In this manner, power supply 25, the operation of which may be controlled by controller 21, may be used to power the operation of water electrolyzer 41 at a range of currents and voltages and may be used to power water electrolyzer 41 continuously or intermittently.

As can also be seen in FIG. 1, water electrolyzer 41 and water reservoir 29 may be coupled together in such a way that (i) water reservoir 29 may be used to supply water to cathode 49 of water electrolyzer 41 and (ii) cathode 49 may be used to supply hydrogen gas to the volume of water contained in water reservoir 29 for mixing with the oxygen gas dispersed in said volume of water. In the present embodiment, such a coupling of water electrolyzer 41 and water reservoir 29 may involve integrating water electrolyzer 41 and water reservoir 29 into an electrolyzer/reservoir assembly 80, wherein at least a portion of water electrolyzer 41, such as cathode 49, may be positioned within the volume of water that is disposed in water reservoir 29.

As can be appreciated, operation of system 11 may result in some depletion of the water present in water reservoir 29, some of said depletion occurring as a result of the electrolysis of water in water electrolyzer 41 and some of said depletion occurring as the humidification of the mixed gas leaving water reservoir 29. To ensure an adequate amount of water remains in water reservoir 29, system 11 may further comprise a water supply (not shown) operatively coupled to water reservoir 29 to replenish the water that is depleted. Additionally or alternatively, water reservoir 29 may simply be replaced when the depletion of water reaches a certain point.

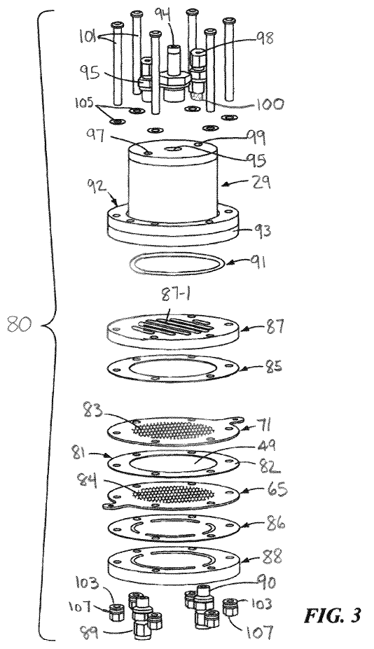

Referring now to FIG. 3, an embodiment of electrolyzer/reservoir assembly 80 is shown in greater detail. As can be seen, electrolyzer/reservoir assembly 80 may comprise a membrane electrode assembly 81. Membrane electrode assembly 81, in turn, may comprise cathode 49, a seal gasket 82, and an anode (which is not shown in FIG. 3 but is positioned opposite cathode 49 on the underside of seal gasket 82). Electrolyzer/reservoir assembly 80 may further comprise cathode collector 71 and anode collector 65. Cathode collector 71, which may have a porous central region 83 defining a fluid diffusion chamber for water access/hydrogen release, may be positioned against the top surface of cathode 49, and anode collector 65, which may have a porous central region 84 defining a fluid diffusion chamber for oxygen release, may be positioned against the bottom surface of the anode.

Electrolyzer/reservoir assembly 80 may further comprise an annular cathode seal gasket 85 and an anode seal gasket 86. Cathode seal gasket 85 may be positioned against the top surface of cathode collector 71, and anode seal gasket 86 may be positioned against the bottom surface of anode collector 65. Electrolyzer/reservoir assembly 80 may further comprise a cathode endplate 87 and an anode endplate 88. Cathode endplate 87, which may comprise a porous central region 87-1 for water access/hydrogen release, may be positioned against cathode seal gasket 85, and anode endplate 88 may be positioned against anode seal gasket 86.

Electrolyzer/reservoir assembly 80 may further comprise oxygen outlet ports 89 and 90, which may be mechanically coupled to anode endplate 88 and fluidly coupled to the anode.

Electrolyzer/reservoir assembly 80 may further comprise water reservoir 29, which may be positioned over cathode endplate 87. A water reservoir seal O-ring 91 may be seated on cathode endplate 87 and may be used to form a fluid seal for the water passing from reservoir 29 to porous central region 87-1 of cathode endplate 87. A reservoir clamp 92 may be mounted on a reservoir sealing plate 93 of water reservoir 29.

Electrolyzer/reservoir assembly 80 may further comprise a reservoir outlet fitting 94, which may be coupled to reservoir 29 through a reservoir outlet port 95, a reservoir water fill fitting 95, which may be coupled to reservoir 29 through a reservoir water fill port 97, and a reservoir oxygen inlet fitting 98, which may be coupled to reservoir 29 through a reservoir oxygen inlet port 99. A sparger 100 may be coupled to the outlet end of reservoir oxygen inlet fitting 98.

Electrolyzer/reservoir assembly 80 may further comprise hardware for mechanically coupling together many of the above-described components. Such hardware may comprise a plurality of screws 101 for assembly clamping, as well as corresponding pluralities of washers 103, washers 105 and nuts 107.

Referring back now to FIG. 1, through the operation of water electrolyzer 41, oxygen gas may be generated at anode 47, and hydrogen gas may be generated at cathode 49. The oxygen gas that is generated at anode 47 may be vented from system 11 to the environment. (Such venting may be effected via a fluid conduit 118 that is fluidly coupled at one end to the output of anode 47.) By contrast, the hydrogen gas that is generated at cathode 49 may be directly added to the volume of water that is contained within water reservoir 29. Such hydrogen gas may then mix, in the volume of water contained within water reservoir 29, with the delivered oxygen gas via inlet 35 and any other gases that have been added to the volume of water within water reservoir 29. The thus-mixed gases may then exit water reservoir 29 through outlet 33.

System 11 may further comprise a hydrogen gas sensor 111. Hydrogen gas sensor 111, which may be conventional, may be fluidly coupled to water reservoir 29 through a fluid conduit 113. Fluid conduit 113, which may consist of or comprise a tube or other structure well-suited for conveying one or more fluids axially therethrough, may have an inlet end fluidly coupled to outlet 33 of water reservoir 29 and an outlet end coupled to an inlet of hydrogen gas sensor 111. Hydrogen gas sensor 111 may also be electrically coupled to controller 21 via an electrical conduit 115, which may comprise a wire or similarly suitable structure. In the present embodiment, electrical conduit 115 may be electrically coupled at a first end to hydrogen gas sensor 111 and at a second end to controller 21. In this manner, depending on the hydrogen gas concentration of the hydrogen/oxygen-containing gas mixture that is sensed by hydrogen gas sensor 111, controller 21 may correspondingly alter the output of power supply 25, which may, in turn, alter the output of water electrolyzer 41. Consequently, hydrogen gas sensor 111 may serve to provide feedback control of the hydrogen gas concentration of the gas mixture. Additionally or alternatively, hydrogen gas sensor 111 may provide confirmation that the hydrogen gas concentration is within a desired range.

System 11 may further comprise a fluid conduit 117. Fluid conduit 117, which may consist of or comprise a tube or other structure well-suited for conveying one or more fluids axially therethrough, may have an inlet end and an outlet end. The inlet end may be fluidly coupled to hydrogen gas sensor 111. The outlet end of fluid conduit 117 may be used to deliver the gas mixture to a desired destination. For example, where system 11 is a subsystem of a ventilation or respiration system, the outlet end of fluid conduit 117 may be coupled to the inlet of a breathing mask or the like.

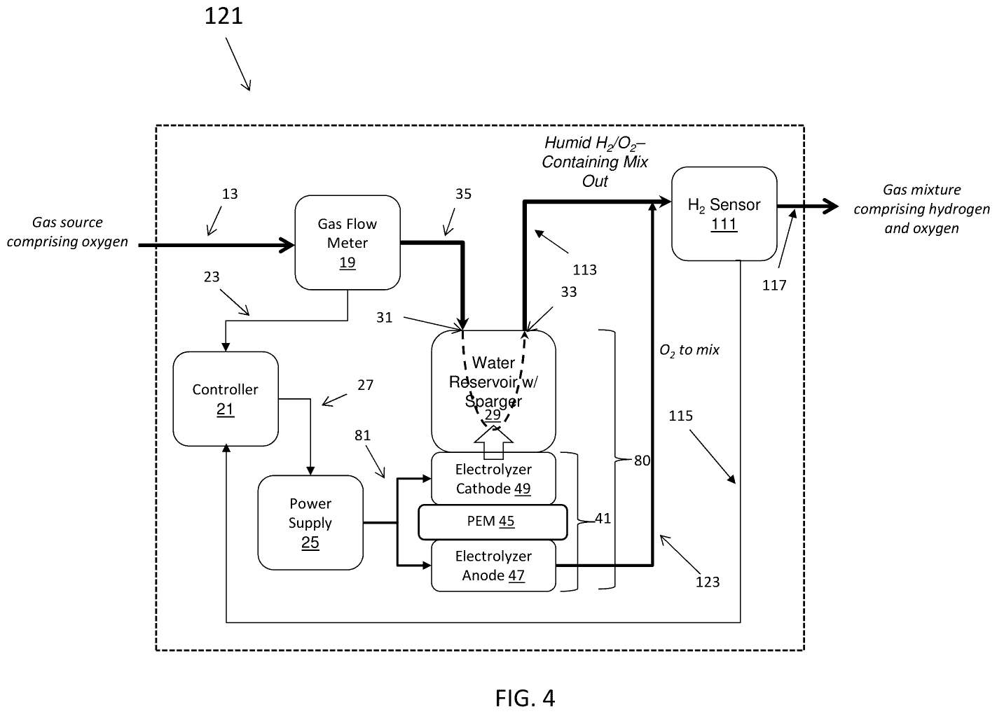

Referring now to FIG. 4, there is shown a simplified schematic representation of a second embodiment of a system for generating a gas mixture, the system being constructed according to the present invention and being represented generally by reference numeral 121. Details of system 121 that are discussed elsewhere in this application or that are not critical to an understanding of the invention may be omitted from FIG. 4 or the accompanying description herein or may be shown in FIG. 4 and/or described herein in a simplified manner.

System 121 may be similar in many respects to system 11. One difference between system 121 and system 11 may be that, whereas system 11 may be configured to vent to the environment the oxygen gas that is generated at anode 47, system 121 may instead add such oxygen gas to the hydrogen/oxygen gas mixture that has left water reservoir 29. To this end, system 121 may include a fluid conduit 123. Fluid conduit 123, which may consist of or comprise a tube or other structure well-suited for conveying one or more fluids axially therethrough, may have an inlet end fluidly coupled to the output of anode 47 and an outlet end fluidly coupled to fluid conduit 113.

As can be appreciated, in another embodiment (not shown), instead of adding the oxygen gas that is generated at anode 47 directly to the hydrogen/oxygen mixture that has already left water reservoir 29, as in the case of system 121, such oxygen gas may be added to the volume of water that is disposed within water reservoir 29. In either case, whether the oxygen that is being added is added to the hydrogen/oxygen mixture that has already left water reservoir 29 or is added to the volume of water that is disposed within water reservoir 29, the addition of such oxygen may counteract the dilution of oxygen that may occur when using system 121.

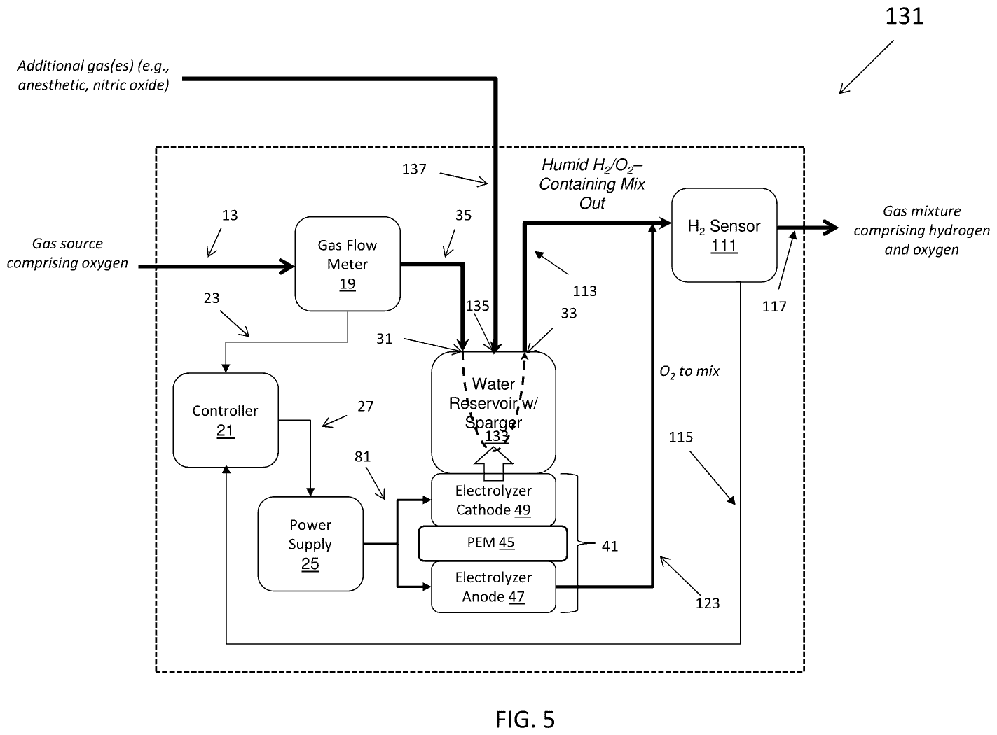

Referring now to FIG. 5, there is shown a simplified schematic representation of a third embodiment of a system for generating a gas mixture, the system being constructed according to the present invention and being represented generally by reference numeral 131. Details of system 131 that are discussed elsewhere in this application or that are not critical to an understanding of the invention may be omitted from FIG. 5 or the accompanying description herein or may be shown in FIG. 5 and/or described herein in a simplified manner.

System 131 may be similar in many respects to system 121. One difference between system 131 and system 121 may be that, whereas system 121 may comprise a water reservoir 29 configured to comprise inlet 31 for receiving an oxygen-containing gas, system 131 may comprise a water reservoir 133 configured to additionally comprise an inlet 135 for receiving one or more additional gases, such as, but not limited to, a therapeutic gas (e.g., anesthesia, nitric oxide). To this end, system 131 may include a fluid conduit 137. Fluid conduit 137, which may consist of or comprise a tube or other structure well-suited for conveying one or more fluids axially therethrough, may have an inlet end fluidly coupled to a gas source and an outlet end fluidly coupled to inlet 135. Preferably, the one or more additional gases delivered to water reservoir 133 are dispensed below the water line of the volume of water disposed in water reservoir 133. In this manner, the mixing of the additional gases with the hydrogen gas and the oxygen gas that are dispersed within the volume of water may be facilitated such that the gas mixture exiting water reservoir 133 may contain a mixture of hydrogen gas, oxygen gas, and the one or more additional gases.

As can be appreciated, the additional gases in fluid conduit 137 may alternatively join the system of 131 via fluid conduit 13 and/or fluid conduit 35.

As can also be appreciated, system 131 may be modified so that the oxygen gas that is added to the hydrogen-containing gas mixture may instead be vented to the environment.

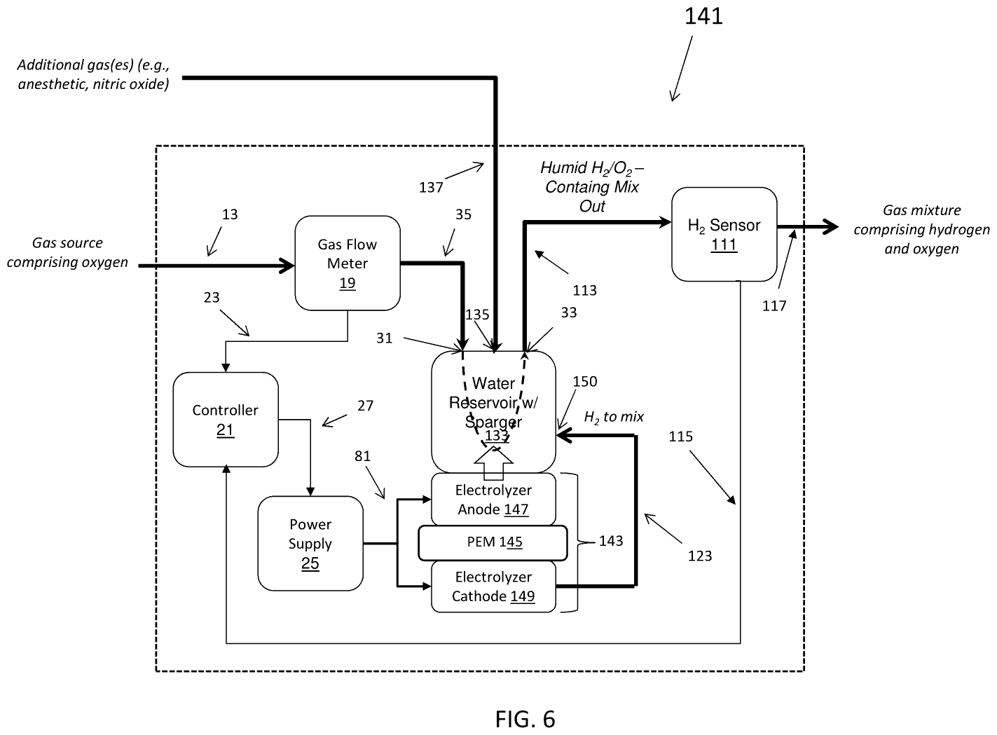

Referring now to FIG. 6, there is shown a simplified schematic representation of a fourth embodiment of a system for generating a gas mixture, the system being constructed according to the present invention and being represented generally by reference numeral 141. Details of system 141 that are discussed elsewhere in this application or that are not critical to an understanding of the invention may be omitted from FIG. 6 or the accompanying description herein or may be shown in FIG. 6 and/or described herein in a simplified manner.

System 141 may be similar in many respects to system 131. One difference between system 141 and system 131 may be that, whereas system 131 may comprise a water electrolyzer 41, system 141 may instead comprise a water electrolyzer 143. Water electrolyzer 143 may be similar in many respects to water electrolyzer 41 and may comprise a polymer electrolyte membrane 145 that may be similar to polymer electrolyte membrane 45, an anode 147 that may be similar to anode 47, and a cathode 149 that may be similar to cathode 49. However, one notable difference between water electrolyzer 143 and water electrolyzer 141 may be that, in water electrolyzer 143, anode 147 may be positioned above polymer electrolyte membrane 145, and cathode 149 may be positioned below polymer electrolyte membrane 145 whereas, by contrast, in water electrolyzer 41, cathode 49 may be positioned above polymer electrolyte membrane 45, and anode 47 may be positioned below polymer electrolyte membrane 45. As a result, in system 141, anode 147 may be positioned within the volume of water disposed in water reservoir 133 and may be used to deliver oxygen gas directly into said volume of water, wherein said oxygen gas may mix with the oxygen gas delivered by fluid conduit 35 and the additional one or more gases delivered by fluid conduit 137. Moreover, cathode 149 may be positioned outside the volume of water disposed in water reservoir 133; consequently, the hydrogen gas that is generated at cathode 149 may not be delivered directly to water reservoir 133. Instead, such hydrogen gas may be added to the volume of water that is disposed within water reservoir 133 at an inlet 150.

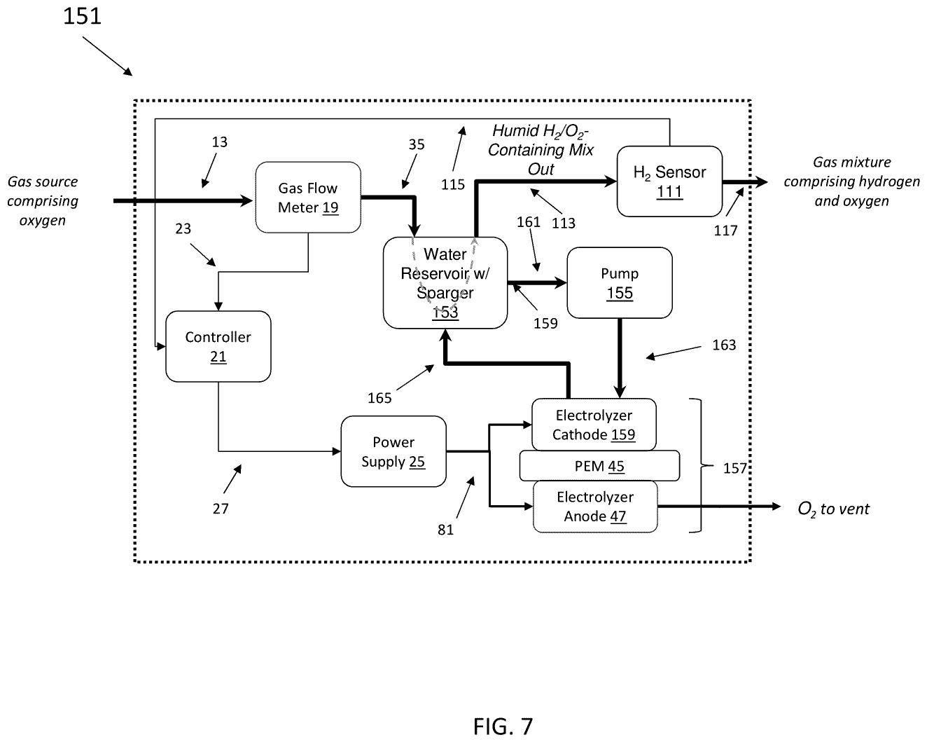

Referring now to FIG. 7, there is shown a simplified schematic representation of a fifth embodiment of a system for generating a gas mixture, the system being constructed according to the present invention and being represented generally by reference numeral 151. Details of system 151 that are discussed elsewhere in this application or that are not critical to an understanding of the invention may be omitted from FIG. 7 or the accompanying description herein or may be shown in FIG. 7 and/or described herein in a simplified manner.

System 151 may be similar in many respects to system 11. For example, like system 11, system 151 may comprise a fluid conduit 13, a gas flow meter 19, a controller 21, an electrical conduit 23, a power supply 25, an electrical conduit 27, an electrical conduit 81, a hydrogen gas sensor 111, a fluid conduit 113, an electrical conduit 115, and a fluid conduit 117. On the other hand, system 151 may differ from system 11 in that, whereas system 11 may comprise a water reservoir and a water electrolyzer that are integrated into an electrolyzer/reservoir assembly 80, with at least electrolyzer cathode 49 of water electrolyzer 41 positioned within the volume of water in the water reservoir 29, system 151 may comprise a water reservoir and a water electrolyzer that are not integrated in the aforementioned fashion. Instead, system 151 may comprise the combination of a water reservoir 153, a water pump 155, and a water electrolyzer 157, all of which may be physically spaced apart from one another.

More specifically, water reservoir 153 may be generally similar to water reservoir 29 of system 11 but may additionally comprise a water outlet 159. A fluid conduit 161, which may consist of or comprise a tube or other similarly suitable structure adapted for conducting fluid axially therethrough, may be coupled at a first end to water outlet 159 and may be coupled at a second end to an inlet of water pump 155. Water pump 155, which may be conventional, may be used to pump water from water reservoir 153 to the electrolyzer cathode 159 of water electrolyzer 157. To this end, a fluid conduit 163, which may consist of or comprise a tube or other similarly suitable structure adapted for conducting fluid axially therethrough, may be coupled at a first end to an outlet of water pump 155 and may be coupled at a second end to water electrolyzer 157 in such a way as to deliver water to electrolyzer cathode 159. Additionally, a fluid conduit 165, which may consist of or comprise a tube or other similarly suitable structure adapted for conducting fluid axially therethrough, may be coupled at a first end to electrolyzer cathode 159 of water electrolyzer 157 and may be coupled at a second end to a hydrogen gas inlet on water reservoir 153. In this manner, hydrogen gas that may be generated at electrolyzer cathode 159 of water electrolyzer 157 may be delivered to the volume of water in water reservoir 153 for mixing with any delivered gases present in the volume of water.

Power supply 25, the operation of which may be controlled by controller 21, may be used to power the operation of water electrolyzer 157 at a range of currents, voltages and water flow rates and may be used to power water electrolyzer 157 continuously or intermittently.

System 151 may be more desirable than system 11 in some respects. For example, system 11 is configured such that it may rely on the constant flooding of water electrolyzer 41 with a substantially stagnant body of water. As can be appreciated, this may be difficult to achieve in practice. By contrast, system 151 may utilize pump 155, which helps to circulate water over water electrolyzer 157.

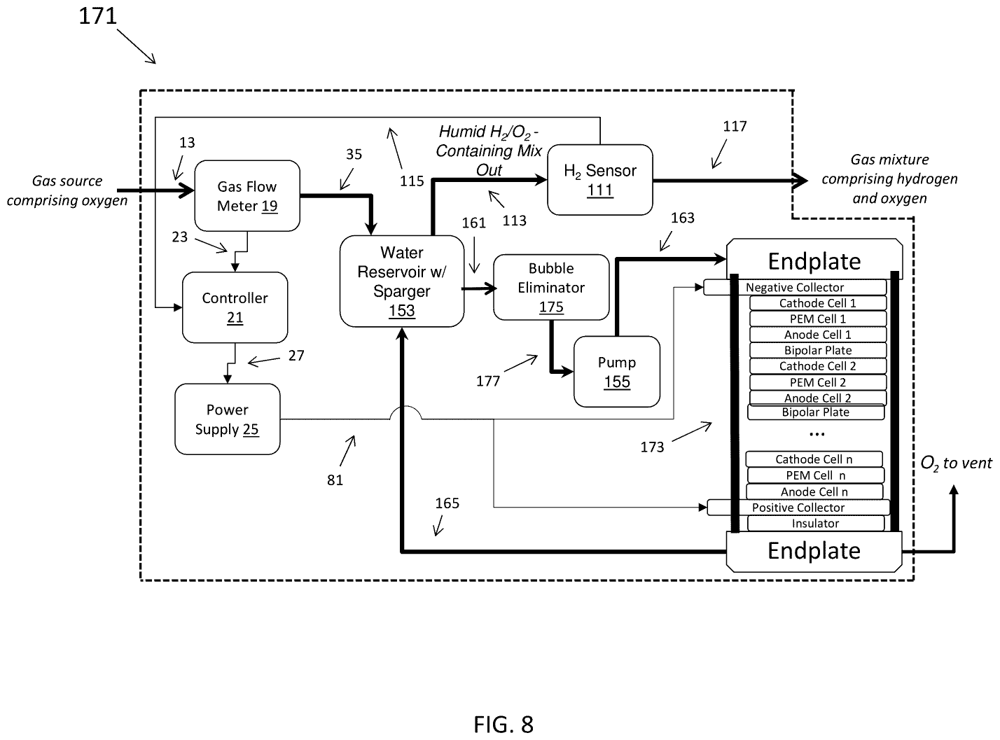

Referring now to FIG. 8, there is shown a simplified schematic representation of a sixth embodiment of a system for generating a gas mixture, the system being constructed according to the present invention and being represented generally by reference numeral 171. Details of system 171 that are discussed elsewhere in this application or that are not critical to an understanding of the invention may be omitted from FIG. 8 or the accompanying description herein or may be shown in FIG. 8 and/or described herein in a simplified manner.

System 171 may be similar in many respects to system 151. One difference between system 171 and system 151 may be that, whereas system 151 may comprise a water electrolyzer 157 comprising a single electrolysis cell, system 171 may comprise a water electrolyzer 173 comprising a stack of electrolysis cells. As can be appreciated, a stack of electrolysis cells may be capable of producing greater volumes of gases than a single cell, particularly without greatly increasing the footprint of the water electrolyzer. This may make system 171 more useful in the higher flow rate respiratory systems in common hospital use, where more hydrogen volume is needed for treatment. It would be difficult to include such a stack in water reservoir 153, itself, since it would be difficult to flood, to the extent needed, each of the cathodes in such a stack. This problem may be ameliorated in system 171 by the provision of a pump that feeds water to the various cathodes in the stack.

Another difference between system 171 and system 151 may be that, whereas system 151 may feed water directly from water reservoir 153 to pump 155 via fluid conduit 161, system 171 may additionally include a bubble eliminator 175 that may be positioned between water reservoir 153 and pump 155. Bubble eliminator 175, which may be conventional, may reduce or minimize the presence of bubbles in the water being supplied to pump 155, which bubbles may interfere with the operation of pump 155. In this manner, by providing bubble eliminator 175, water may be conveyed from water reservoir 153 to bubble eliminator 175 via fluid conduit 161, and, thereafter, may be conveyed from bubble eliminator 175 to pump 155 via a fluid conduit 177 (wherein fluid conduit 177 may be similar in structure and function to fluid conduit 161), with the presence of bubbles in the water reaching pump 155 being reduced or minimized.

It is to be understood that systems 151 and 171 may be modified so that the oxygen that is produced at the one or more anodes may be added to the volume of water in the water reservoir or to the gas mixture after it has left the water reservoir. Additionally or alternatively, systems 151 and 171 may be modified so that one or more additional gases, such as anesthesia or nitric oxide, may be added to the water reservoir 153 directly or by introduction to fluid conduit 13 or 35.

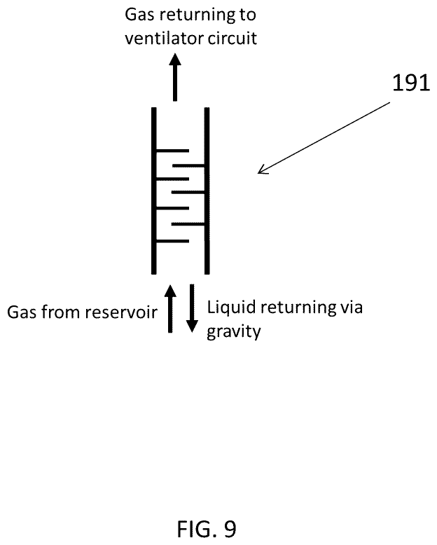

As can be appreciated, while it may be desirable for the mixed oxygen and hydrogen gases leaving the water reservoir to draw a certain amount of water vapor from the water reservoir, it may be undesirable for the amount of water vapor to exceed such an amount or to leave in the liquid state. Accordingly, referring now to FIG. 9, there is schematically shown one embodiment of a baffled fluid conduit of the type that may be positioned at the gas outlet of the water reservoir according to the present invention, said baffled fluid conduit being represented generally by reference numeral 191. Baffled fluid conduit 191 may be designed to return liquid to the water reservoir via gravity and to allow the humidified gas phase to exit, for example, for administration to a patient or to some other destination.

As noted above, the present invention is also directed at a system in which the above-described dilute mixtures of gases may be delivered to a person for therapeutic applications using equipment, such as, but not limited to, ventilators, oxygen face masks and cannulas, anaesthesia machines, respirators, high flow respirators, and extracorporeal membrane oxygenators (ECMO).

Existing ventilator systems for respiratory therapy typically come in a wide variety of configurations but generally include the same functions. More specifically, generally speaking, an oxygen-rich gas supply, such as pure medical grade oxygen, air, mixes thereof or mixes with other gases, is connected to a ventilator. The ventilator is connected to a circuit tubing system which delivers the respiratory gas flow to and receives from the patient's respiratory equipment during breathing, and actively delivers oxygen-rich gas, with or without an integral humidifier, to the circuit. The delivered gas may be partially recirculated, in which case the ventilator exhausts a portion of the return (expired) gas, and may actively reduce carbon dioxide in the recirculated portion using a scrubber. The gas delivery rate from the ventilator may be governed by a variety of pressure, volume or flow control algorithms, all driven by patient-induced breathing rhythms, involving oxygen-rich gas supply flow, ventilator pump flow, circuit flow control valves, and feedback from pressure, flow, blood oxygen and/or other sensors. The circuit may be fitted with heating systems to prevent condensation of humidified gas, which is typically regulated to a dewpoint near normal physiological temperature (˜35° C.). A more detailed review of ventilator applications and types may be found, for example, in Tài Pham, Laurent J. Brochard, Arthur S. Slutsky, “Mechanical Ventilation: State of the Art,” Mayo Clinic Proceedings, Volume 92, Issue 9, 2017, Pages 1382-1400; U.S. Pat. No. 10,821,259 B2, inventor Borrello, issued Nov. 3, 2020; and U.S. Pat. No. 11,247,016 B2, inventor Novkov, Feb. 15, 2022, all of which are incorporated herein by reference.

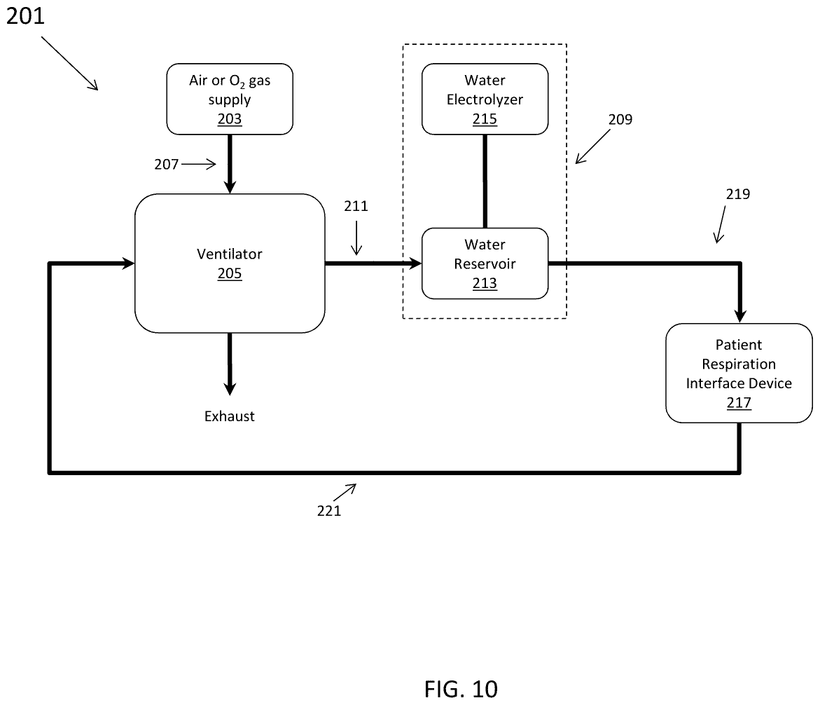

Referring now to FIG. 10, there is shown a simplified schematic representation of a first embodiment of a ventilator circuit, the system being constructed according to the present invention and being represented generally by reference numeral 201. Details of system 201 that are discussed elsewhere in this application or that are not critical to an understanding of the invention may be omitted from FIG. 10 or the accompanying description herein or may be shown in FIG. 10 and/or described herein in a simplified manner.

System 201 may comprise an air or oxygen gas supply 203, which may be, for example, one or more gas tanks containing air, oxygen-enriched air, or pure oxygen. System 201 may further comprise a ventilator 205, which may be conventional. A fluid conduit 207, which may be a tube or similarly suitable structure configured to convey one or more fluids axially therethrough, may have a first end fluidly coupled to an outlet of air or oxygen gas supply 203 and may have a second end fluidly coupled to an inlet of ventilator 205.

System 201 may further comprise a hydrogen generating/mixing subsystem 209, which may be used to generate hydrogen gas, preferably via the electrolysis of water, and to mix such hydrogen gas with the air or oxygen gas supplied from ventilator 205. Hydrogen generating/mixing subsystem 209 may be fluidly coupled to ventilator 205 via a fluid conduit 211, wherein fluid conduit 211 may be similar in structure and function to fluid conduit 207. Hydrogen generating/mixing subsystem 209 may comprise the combination of a water reservoir 213 and a water electrolyzer 215. As can be appreciated, hydrogen generating/mixing subsystem 209 may comprise a system like one of systems 11, 121, 131, 141, 151, and 171. More specifically, water reservoir 213 may comprise a water reservoir that is similar or identical to one of water reservoirs 29, 133 and 153, and water electrolyzer 215 may comprise a water electrolyzer that is similar or identical to a corresponding one of water electrolyzers 41, 143, 157 and 173.

System 201 may further comprise a patient respiration interface device 217, which may be, for example, a mask, a cannula or other suitable respiratory equipment of the type conventionally used to administer gases to a patient via inhalation. Device 217 may be fluidly coupled to hydrogen generating/mixing subsystem 209 via a fluid conduit 219, which may be similar in structure and function to fluid conduit 207.

System 201 may further comprise a fluid conduit 221. Fluid conduit 221, which may be similar in structure and function to fluid conduit 207, may be used to convey exhaled gases from a patient via device 217 to ventilator 205.

As can be appreciated, because hydrogen generating/mixing subsystem 209 produces a humidified gas, hydrogen generating/mixing subsystem 209 may obviate the need for a dedicated humidifier in system 201.

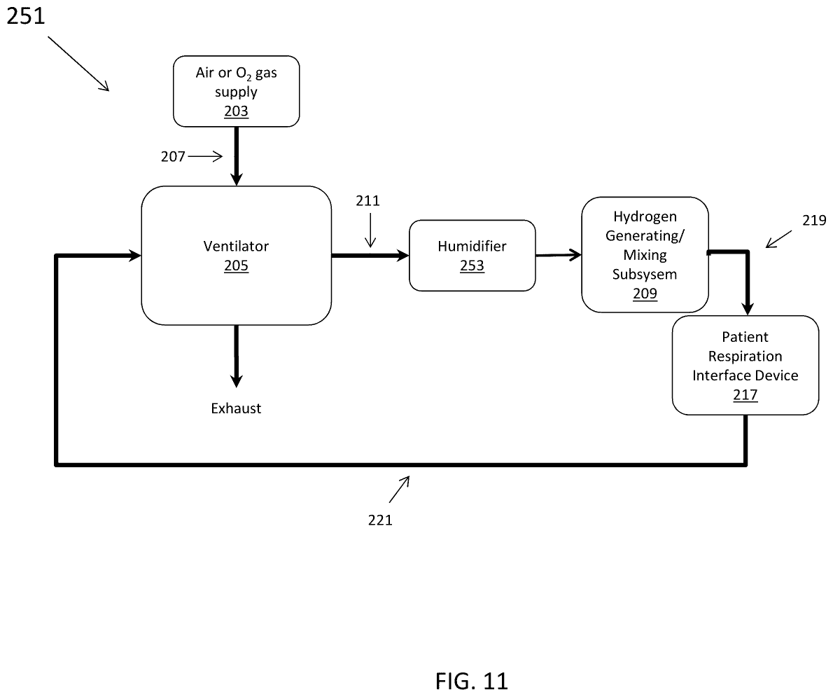

Referring now to FIG. 11, there is shown a simplified schematic representation of a second embodiment of a ventilator circuit, the system being constructed according to the present invention and being represented generally by reference numeral 251. Details of system 251 that are discussed elsewhere in this application or that are not critical to an understanding of the invention may be omitted from FIG. 11 or the accompanying description herein or may be shown in FIG. 11 and/or described herein in a simplified manner.

System 251 may be similar in many respects to system 201. One difference between system 251 and system 201 may be that system 251 may further comprise a humidifier 253, which may be similar to a conventional humidifier of the type commonly found in a ventilation system. Humidifier 253 may be positioned in series before hydrogen generating/mixing subsystem 209 and may be used to provide additional humidification so that the mixed gas delivered to a patient has a desired level of humidification.

The following examples are given for illustrative purposes only and are not meant to be a limitation on the invention described herein or on the claims appended hereto.

### Example 1

A single-cell water electrolyzer (40-cm² active area comprising 150 μm thick perfluorosulfonic acid proton-exchange membrane (equivalent weight of dry polymer approximately 1,100 g per mole of sulfonic acid groups), iridium anode catalyst against porous titanium collector and platinum cathode catalyst against porous carbon collector) was built having the general construction disclosed in U.S. Pat. No. 10,091,985 B2, inventors Tempelman et al., issued Oct. 9, 2018, which is incorporated herein by reference, but with anode and cathode electrode formulations swapped, and including an integrated water electrolyzer (approximately 500 cc volume) in direct contact with and above the cathode collector, per the configuration of FIG. 1, but without any controller driving production. Electrolyzer current and oxygen gas flow setpoints were governed by the operator (hand calculation of Faraday's Law and manual current setpoint to power supply based on oxygen controller flow setpoint). Titanium plates were used to clamp the hardware together to approximately 2000 lbs (to achieve fluidic seals, to maintain flow paths specified by flow fields and to ensure appropriate contact pressures for electrical conductivity and membrane mechanical support), the cathode side plate interposing the cathode collector and the reservoir and having large slots to allow for facile fluid communication at the bottom of the reservoir. An O-ring seal retained the water in the reservoir, and the reservoir had three ports at the top face for oxygen inlet, oxygen outlet and temperature probing, respectively. The oxygen inlet was terminated with a plastic sparger, and the electrolyzer reservoir was filled with deionized water. The anode outlet was vented directly to the atmosphere. Current was ramped up to 0.4 A, 1.6 A, 2.7 A and 5.4 A (˜138 mA/cm²) over 70 min. The water temperature in the electrolyzer did not warm and, instead, cooled noticeably from 23.0° C. to 21.7° C. during ˜2 hr. operation at up to 5.5 A (138 mA/cm²); this suggests that the latent cooling power of humidifying the dry oxygen was greater than the ohmic heating power of the operating electrolyzer. The setup was able to yield hydrogen-oxygen mixtures reliably and safely at 0.25-2.2 vol % at 1.0 SLPM (standard liter per minute) and 2.0 SLPM. The electrolyzer operated at high efficiency and with no apparent mass transfer limitation at up to 138 mA/cm².

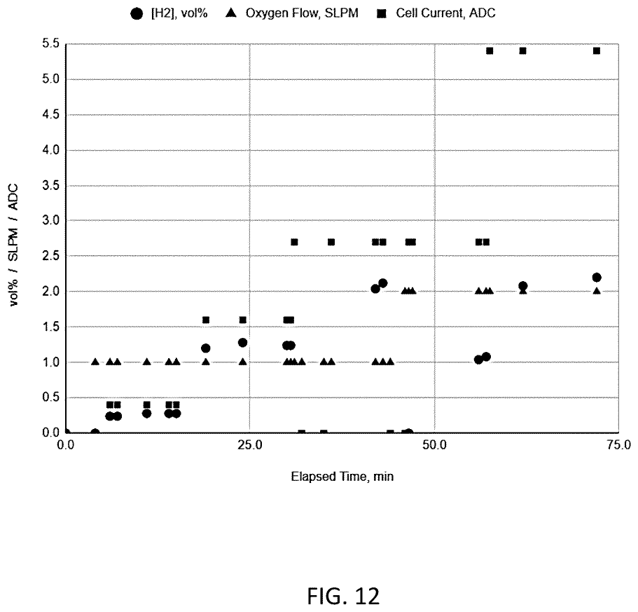

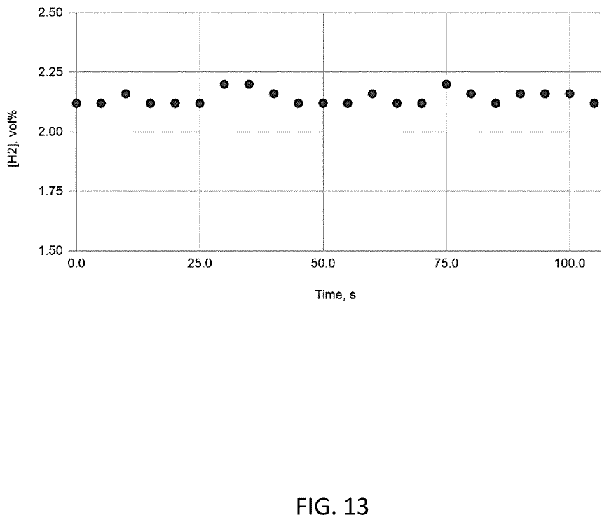

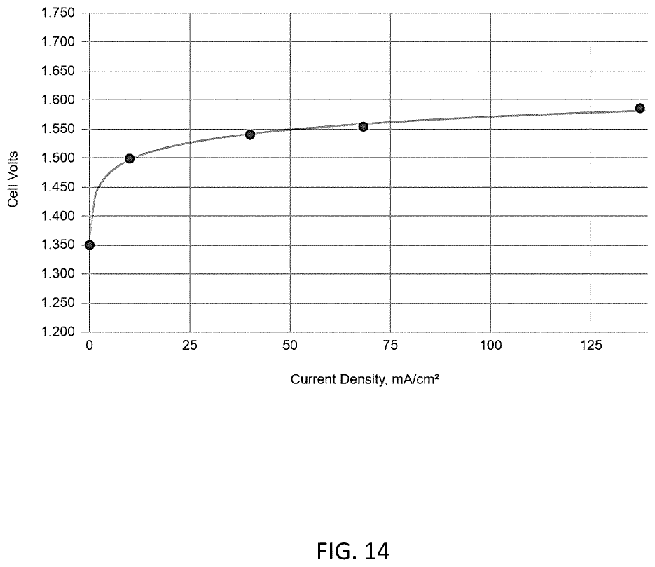

FIG. 12 is a timeplot of the hydrogen concentration, oxygen flow rate, and cell current during constant-current electrolysis of water using the above-described setup. FIG. 13 is a graph showing the measured hydrogen concentration at the reservoir outlet during constant electrolysis at 2.7 A and 1.0 SLPM sparged oxygen delivery to electrolyzer reservoir operating the above-described setup. FIG. 14 is a polarization plot operating the above-described setup.

### Example 2

A three-cell, bipolar water electrolyzer (50-cm² active area comprising 150 μm thick, 1,100 equivalent weight perfluorosulfonic acid proton-exchange membrane, iridium anode catalyst against porous titanium collector and platinum cathode catalyst against porous carbon collector) is constructed in a manner similar to those known in the art. Titanium or stainless steel plates are used to clamp the hardware together (sufficiently to achieve fluidic seals, to maintain flow paths specified by flow fields and to ensure appropriate contact pressures for electrical conductivity and membrane mechanical support), the bipolar separator plates interposing the cells being constructed from titanium and, optionally, bearing a conductive coating for decreasing contact resistance and oxidation. The end plates and bipolar separators have ports and manifolds for fluidic pathways for water feed and hydrogen and oxygen gas removal among inlet and outlet pathways at the edge of each cell active area. While the cells are electronically and electrolytically connected in series, they are fluidically plumbed in parallel. End plates or, optionally, end separators are utilized for connection to a direct current source. Flow fields are inserted between the separator and collector of each anode and cathode to provide compressive forces and conductivity for low cell resistance, as well as defined channels for uniform fluid delivery. The anode outlet is vented directly to the atmosphere, releasing electrolytically produced oxygen and some water vapor. With sufficient supply of pure water and a means of temperature management, the electrolyzer can sustain high currents (i.e. 30 A or more) and, thereby, produce oxygen and hydrogen efficiently for long durations of continuous or discontinuous operation. A heat exchanger is optionally placed between the water pump and the electrolyzer inlet for warming or cooling the system to the desired humidifier temperature.

The 3-cell bipolar electrolyzer medical oxygen feed flows into a sparger in a separate humidifier reservoir, and the water in this humidifier reservoir is recirculated across the electrolyzer cathodes using a pump interposed between the elements. With oxygen feed to the humidifier sparger at 20 SLPM, applying a current of 18.3 ADC will result in a humidifier outlet stream composed of humid oxygen with 2 vol-% hydrogen.

Additional advantages, aspects and features that may be applicable to one or more embodiments of the present invention include the following:
One or more embodiments of the present invention may be used to create a hydrogen-oxygen or hydrogen-air gas mixture of a desired composition, which gas mixture preferably has a hydrogen concentration below the lower explosive limit and more preferably has a hydrogen concentration no greater than 4 vol % hydrogen in air or oxygen, and even more preferably has a hydrogen concentration of about 0.5-2 vol % hydrogen in air or oxygen. Such a gas mixture may also have an oxygen concentration of about 20 vol % to slightly less than 100 vol % (e.g., 99.95 vol %).
In one or more embodiments of the present invention, the system may be configured to add 1.0-2.4 SLPM of hydrogen to 60 SLPM of air or oxygen or the corresponding amounts of hydrogen to lesser amounts of air or oxygen to constitute up to 4 vol % volume percent hydrogen.
One or more embodiments of the present invention may be applicable to invasive (i.e., for intubated critical care applications) and non-invasive ventilator types, and where the humidifier may be further integrated within the ventilator product, the subject invention can be included within the ventilator product specification.
The mixing of hydrogen and oxygen gases in water in accordance with the present invention reduces the likelihood that these gases will react causing a safety hazard of explosion or flame.
In at least one embodiment, an aspect of the present invention is a system comprising a water electrolyzer, a cathode feed pump and a recirculated water reservoir with oxygen sparger for generation of hydrogen-oxygen mixtures under constant oxygen flow and electrolyzer current operation.
In at least one embodiment, an aspect of the present invention is a system comprising a water electrolyzer and a cathode-contacting water reservoir with oxygen sparger for generation of hydrogen-oxygen mixtures under constant oxygen flow and electrolyzer current operation.
In at least one embodiment, the water reservoir serves at least two purposes, namely, (i) providing a supply of water to be electrolyzed by the water electrolyzer; and (ii) providing a medium in which the oxygen and hydrogen gases may mix safely. Mixing may also occur in the headspace above the water in the reservoir, but such mixing in the headspace may be less safe than in water.
The present invention may be used to generate gas mixtures that may be used to treat certain medical conditions including, but not limited to, ischemia-reperfusion injury, particularly ischemia-reperfusion injury to brain tissue. More generally, there are many medical indications where H₂ may possibly be beneficial in mitigating tissue injury including ischemic stroke, cardiac arrest, heart myocardial infarction, surgeries utilizing cardiac bypass, traumatic brain injury, concussion, cerebral palsy and other indications where the mode of damage may be related to ischemia reperfusion injury that results from oxidative or inflammatory damage. Ischemia reperfusion injury can occur when blood is blocked from almost any organ including the heart, brain, bowels, limb and skin. The present invention may be used in indications where the mode of damage and the mode of protection or repair is not specifically or exactly known, such as an adjunct to other cancer treatments, for sudden hearing loss, kidney disease or injury. The present invention may be used to treat any or all of these condition, as well as others.

The embodiments of the present invention described above are intended to be merely exemplary and those skilled in the art shall be able to make numerous variations and modifications to it without departing from the spirit of the present invention. All such variations and modifications are intended to be within the scope of the present invention.

What is claimed is:

1. A system for generating a gas mixture, the system comprising:
a water reservoir, the water reservoir comprising a volume of water, a first gas inlet through which a first delivered gas is delivered to the volume of water, and a gas outlet through which a gas mixture exits the water reservoir; and
a water electrolyzer, the water electrolyzer coupled to the water reservoir to receive water from the volume of water and to output a first generated gas that is added to the volume of water;
whereby the first delivered gas and the first generated gas mix in the volume of water to form the gas mixture and exit the water reservoir through the gas outlet.

2. The system as claimed in claim 1 wherein the first delivered gas is oxygen and wherein the first generated gas is hydrogen.

3. The system as claimed in claim 1 wherein the first delivered gas is one of air and oxygen-enriched air and wherein the first generated gas is hydrogen.

4. The system as claimed in claim 1 wherein at least a portion of the water electrolyzer is positioned within the volume of water in the water reservoir.

5. The system as claimed in claim 4 wherein the first generated gas is hydrogen and wherein the water electrolyzer comprises a cathode, the cathode being positioned within the volume of water in the water reservoir to output hydrogen directly into the volume of water.

6. The system as claimed in claim 5 wherein the water electrolyzer further comprises an anode for outputting a second generated gas and wherein the system further comprises a first fluid conduit for venting the second generated gas.

7. The system as claimed in claim 5 wherein the water electrolyzer further comprises an anode for outputting a second generated gas and wherein the system further comprises a second fluid conduit for conveying the second generated gas to the gas mixture at a location outside of the water reservoir.

8. The system as claimed in claim 1 wherein the water electrolyzer is positioned entirely outside of the water reservoir.

9. The system as claimed in claim 8 further comprising a pump, the pump being fluidly coupled between the water reservoir and the water electrolyzer to pump water from the water reservoir to the water electrolyzer.

10. The system as claimed in claim 9 wherein the pump is a cathode feed pump.

11. The system as claimed in claim 9 wherein the water electrolyzer consists of a single electrolysis cell.

12. The system as claimed in claim 9 wherein the water electrolyzer comprises a bipolar stack of electrolysis cells.

13. The system as claimed in claim 12 further comprising a bubble eliminator, the bubble eliminator being positioned in series between the water reservoir and the pump.

14. The system as claimed in claim 1 wherein the water electrolyzer comprises a polymer electrolyte membrane, an anode operatively coupled to one face of the polymer electrolyte membrane, and a cathode operatively coupled to an opposing face of the polymer electrolyte membrane.

15. The system as claimed in claim 1 wherein the water reservoir further comprises a second gas inlet through which a second delivered gas is delivered to the volume of water.

16. The system as claimed in claim 1 wherein the second delivered gas comprises at least one therapeutic gas selected from the group consisting of anesthesia and nitric oxide.

17. The system as claimed in claim 1 wherein the gas mixture comprises hydrogen and oxygen and wherein the hydrogen concentration does not exceed 4 vol %.

18. The system as claimed in claim 1 further comprising a power supply for powering the water electrolyzer and a controller for controlling the output of the power supply.

19. The system as claimed in claim 18 further comprising a gas flow meter for measuring the flow of gas of the first delivered gas to the volume of water, the gas flow meter being operatively coupled to the controller.

20. The system as claimed in claim 18 further comprising a hydrogen gas sensor for measuring the hydrogen concentration of the gas mixture, the hydrogen gas sensor being operatively coupled to the controller.

21. A ventilator system, the ventilator system comprising:
an oxygen-containing gas supply, the oxygen-containing gas supply providing a quantity of an oxygen-containing gas;
a ventilator, the ventilator operatively coupled to the oxygen-containing gas supply to receive the oxygen-containing gas and to output the oxygen-containing gas;
the system for generating a gas mixture as claimed in claim 1, wherein the oxygen-containing gas is the first delivered gas and wherein the first generated gas is hydrogen gas, whereby the gas mixture comprises oxygen and hydrogen; and
a patient respiration interface device, the patient respiration interface device being operatively coupled to the ventilator and to the system for generating a gas mixture so that the gas mixture is administered to a patient via inhalation.

22. A method for generating a gas mixture, the method comprising the steps of:
providing a water reservoir, the water reservoir comprising a volume of water, a first gas inlet through which a first delivered gas is delivered to the volume of water, and a gas outlet through which a gas mixture exits the water reservoir;
providing a water electrolyzer, the water electrolyzer coupled to the water reservoir to receive water from the volume of water and to output a first generated gas that is added to the volume of water;
delivering the first delivered gas to the volume of water;
operating the water electrolyzer to generate the first generated gas;
whereby the first delivered gas and the first generated gas mix in the volume of water to form the gas mixture and exit the water reservoir through the gas outlet.
